(19) United States
(12) Patent Application Publication
(10) Pub. No.: US 2013/0045489 A1
(43) Pub. Date: Feb. 21, 2013

(54) NEURAL NANOPROBES

(71) Applicants: Karl Mendoza, Niles, IL (US); John D. Griffin, Toano, VA (US)

(72) Inventors: Karl Mendoza, Niles, IL (US); John D. Griffin, Toano, VA (US)

(73) Assignee: COLLEGE OF WILLIAM AND MARY, Williamsburg, VA (US)

(21) Appl. No.: 13/370,446

(22) Filed: Feb. 10, 2012

Related U.S. Application Data
(60) Provisional application No. 61/441,324, filed on Feb. 10, 2011.

(51) Int. Cl. C08G 73/04; G01N 21/47; B82Y 5/00

(52) U.S. Cl. 435/7.21; 525/54.1; 977/773; 977/927

(57) ABSTRACT

Neural nanoprobes are described, as well as methods for their use, including for use as a tagging system for neuronal pathway identification. The neural nanoprobes comprise metallic nanoparticles that are conjugated to both (i) a cationic polymer such as polyethylenimine and (ii) an antibody to a vesicular transporter protein. These methods allow retrograde characterization of glutamatergic neurons in a tissue slice preparation. Since the nanoparticles used are non-lipid-soluble and are specifically conjugated to enter and escape the synaptic vesicular machinery, these nanoparticles allow probing of a neuron's somatic origin, via the synapse, by diffusional means.

### CROSS-REFERENCE TO RELATED APPLICATIONS

This application claims the benefit of and priority to U.S. Provisional Patent Application Ser. No. 61/441,324, filed Feb. 10, 2011, the entire disclosure of which is incorporated by reference herein.

### STATEMENT REGARDING FEDERALLY SPONSORED RESEARCH AND DEVELOPMENT

This invention was made with government support under Grants No. 1 R15 NS053794-01 and 1 R15 NS064361-01A1, awarded by the National Institutes of Health. The government has certain rights in the invention.

### FIELD OF INVENTION

The field of the invention relates to nanoprobes and methodology for retrograde labeling of neurons.

### BACKGROUND OF THE INVENTION

Thermoregulatory neurons in the preoptic area of the anterior hypothalamus (“POA”) form synaptic networks that can effect responses that regulate body temperature. Changes in this area's neuronal activity have been correlated with the initiation of thermoregulatory mechanisms (e.g., see Imbery et al., 2008, “The effects of Cirazoline, an alpha-1 adrenoreceptor agonist, on the firing rates of thermally classified anterior”, Brain Res., 1193, p. 93-101). Recent studies also suggest that certain thermoregulatory phenomena, such as hyperthermia, can be elicited by activation of neurons in the dorsomedial hypothalamus (“DMH”) (e.g., see Nakamura and Morrison, 2008, “A thermosensory pathway that controls body temperature”, Nat. Neurosci., 11, p. 62-71). To produce these distinct changes in thermoregulatory control, POA neurons may have direct axonal connections to the DMH.

To determine the thermoregulatory role of POA neurons that project to the DMH, their phenotypes and connectivity must be characterized. Past studies have attempted to phenotype these neurons through their synaptic projections. For example, certain techniques, such as glutamate decarboxylase staining, resulted in localization of not only the synapses of interest but also nearby axonal fibers.

In addition to therapeutic applications as vehicles, nanoparticles may expand bio-imaging techniques as contrast agents, by functionally assessing and localizing specific molecular signatures or physiological systems. Cellular metallic nanoparticle studies are still relatively nascent, and literature involving neuronal applications is still evolving. Previous studies have, for example, used gold nanoparticle vehicles to deliver genetic or drug payloads to the nucleus of a cell (Olivier, J. C., 2005. “Drug transport to brain with targeted nanonparticles”, NeuroRx., 2, p. 108-119). It would be advantageous to reverse that process such that the payload (subsequently referred to as ‘conjugates’) facilitates delivery of the vehicle (i.e., the gold nanoparticle) to the neuronal soma, which would facilitate a potential tagging system for neuronal pathway identification.

Furthermore, since thin neuronal tissue slices remain viable in vitro for approximately 12 hours under proper conditions, an appropriate retrograde labeling technique must work in less time to allow for the targeted electrophysiological recording of these neurons in the POA. Prior art methods exist, but there is a need for a method that simultaneously offers the appropriate time course, specificity, and low biotoxicity required to leave neurons of interest suitably intact for live-cell recording.

### BRIEF SUMMARY OF THE INVENTION

Neural nanoprobes are described, as well as methods for their use, including for use as a tagging system for neuronal pathway identification. The neural nanoprobes comprise metallic nanoparticles that are conjugated to both (i) a cationic polymer and (ii) an antibody to a vesicular transporter protein. These methods allow retrograde characterization of glutamatergic neurons in a tissue slice preparation. Since the nanoparticles used are non-lipid-soluble and are specifically conjugated to enter and escape the synaptic vesicular machinery, these nanoparticles allow probing of a neuron's somatic origin, via the synapse, by diffusional means.

It is an object of the invention to provide neural nanoprobes comprising metallic nanoparticles that are conjugated to both (i) a cationic polymer and (ii) an antibody to a vesicular neurotransmitter transport protein (also called vesicular neurotransmitter transporters, or vesicular transporter proteins).

It is an object of the invention to provide a method for retrograde labeling of neurons comprising: (1) injecting neural nanoprobes into neural tissue, (2) allowing uptake of said neural nanoprobes into synaptic vesicles at axonal terminals, (3) allowing endosomal escape of said neural nanoprobes into the cytosol, and (4) allowing diffusion of said neural nanoprobes from the synaptic terminal region to the neuronal soma, wherein said neural nanoprobes comprise metallic nanoparticles conjugated to both a cationic polymer and an antibody to a vesicular transport protein.

### BRIEF DESCRIPTION OF THE DRAWINGS

The summary above, and the following detailed description, will be better understood in view of the drawings which depict details of preferred embodiments.

FIG. 1 shows a schematic diagram of the biochemical mechanism for uptake and subsequent endosomal escape of neural nanoprobes of the present invention.

FIG. 1A is a schematic diagram depicting probe conjugates, such as VGLUT-2 antibodies (curvy line) and PEI molecules (Y-shape), surface-conjugated on metallic nanoparticles (interior circle). FIG. 1B depicts the conjugates from FIG. 1A facilitating membrane protein attachment during vesicular formation. FIG. 1C is a depiction of endosomal escape by the conjugates from FIG. 1A from the vesicular lumen into the cytosol. FIG. 1D is a schematic diagram of the entire process, including the processes depicted in FIG. 1A, FIG. 1B, and FIG. 1C. Neural nanoprobes are injected into the DMH at left, and on the right is a schematic blow-up showing the various processes depicted in FIG. 1A, FIG. 1B, and FIG. 1C.

FIG. 2 shows a graph comparing the efficacy of Probe A for DMH tissue and non-DMH tissue.

FIG. 3 shows a plot that provides the normalized spatial distribution of Probe A in DMH-injected tissue. The origin (1A) represents the DMH injection site. Higher axis numbers along the y-axis indicate a more dorsal direction, while higher axis letters along the x-axis indicate a more caudal direction. The shading legend reflects the number of labeled neurons observed over a 20-slice average.

FIG. 4 shows a chart comparing POA neuron labeling efficacy as a function of the probe (Probes A, B, C, D, and DX).

FIG. 5 shows a chart comparing POA neuron labeling efficacy as a function of the probe, specifically comparing Probe A (VGLUT-2) to Probe Z (VGAT).

FIG. 6 shows a plot that provides the normalized spatial distribution of Probe Z in DMH-injected tissue. The origin (1A) represents the DMH injection site. Higher axis numbers along the y-axis indicate a more dorsal direction, while higher axis letters along the x-axis indicate a more caudal direction. The oval denotes the POA area. The shading legend reflects the number of labeled neurons observed over a 23-slice average.

### DETAILED DESCRIPTION OF THE INVENTION

The present invention is directed to neural nanoprobes suitable for labeling and/or tracing of neurons.

The neural nanoprobes of the present invention comprise metallic nanoparticles that are conjugated to both (i) a cationic polymer and (ii) an antibody to a vesicular neurotransmitter transport protein (also called vesicular neurotransmitter transporters, or vesicular transporter proteins).

Suitable vesicular neurotransmitter transporters to which said antibodies are directed include proteins that account for the vesicular transport of catecholamines, serotonin, histamine, acetylcholine, GABA, glycine, and glutamate. Representative vesicular neurotransmitter transporter proteins include, but are not limited to: vesicular glutamate transporter proteins (“VGLUT”), vesicular inhibitory amino acid transporters (“VIAAT”) including the vesicular GABA transporter (“VGAT”), and vesicular amine transporters (“VAT”). For example, antibodies to VGLUT-1, VGLUT-2, and/or VGLUT-3, all of which are vesicular glutamate transporter proteins, would be suitable for conjugation to metallic nanoparticles in accordance with the methods described herein.

Antibodies to VGAT are also suitable for use in accordance with the methods of the invention.

Antibodies can be monoclonal or polyclonal antibodies, and mixtures of antibodies are contemplated. In representative embodiments, a single nanoparticle is conjugated to one or more such antibodies. A typical loading comprises one or more antibodies per nanoparticle, typically greater than three conjugated antibodies (either identical or different) per nanoparticle.

The nanoparticles are also conjugated to at least one cationic polymer. In one embodiment, the cationic polymer is polyethylenimine (“PEI”). Typical loading of polyethylenimine is one or more polyethylenimine chains per nanoparticle, sometimes greater than 3 polyethylenimine chains per individual nanoparticle. Other suitable cationic polymers include but are not limited to chitosan and dextran amine.

Suitable nanoparticles for use in the present invention are metallic nanoparticles, for example, silica nanoparticles having a metallic coating. In one embodiment, the nanoparticles are gold nanoparticles. In one such embodiment, the gold nanoparticles (“AuNPs”) are spherical, colloidal nanoparticles composed of a dielectric silica core and an ultra-thin gold coating, providing them with a strong reflectivity (peak wavelength: glutamatergic=524 nm, GABAergic=510 nm), and an extremely low biotoxicity.

Relative to the prior art, the neural nanoprobes of the present invention have one or more distinct advantages in terms of their ability to enter neuronal cells, achieve endosomal escapes, and function as an imageable retrograde labeler and tract tracer for electrophysiological recording.

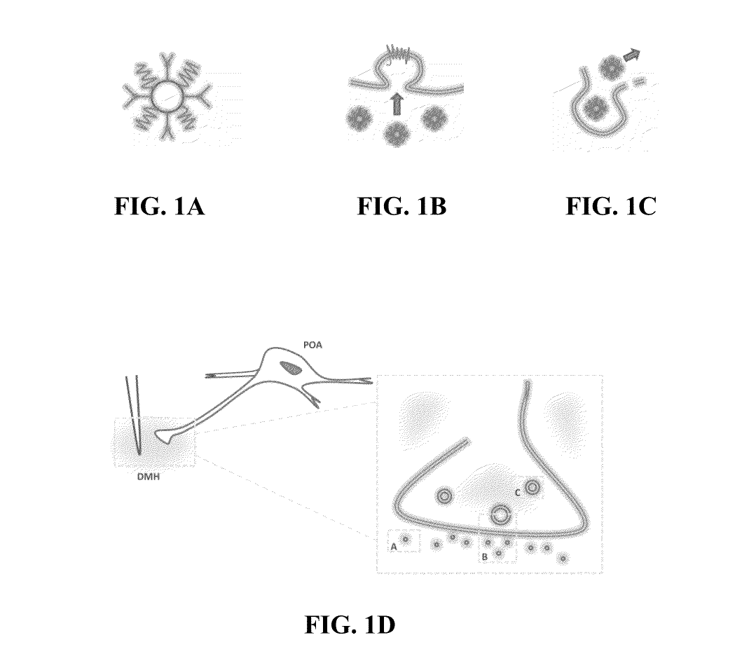

In a representative embodiment of the method of the invention depicted in FIG. 1D, the probe is injected in the DMH to allow vesicular uptake at the axon terminal. In FIG. 1A, a schematic of a probe conjugate is depicted. For example, gold nanoshells (interior circle) can be conjugated to VGLUT-2 antibodies (depicted as wavy lines) and PEI (Y-shape). In FIG. 1B, the conjugates facilitate membrane protein attachment during vesicular formation. In FIG. 1C, endosomal escape is depicted from the vesicular lumen into the cytosol. Intracellular diffusion and potentially retrograde axonal transport machinery facilitation result in nanoaggregate deposition in the neuronal soma.

### EXAMPLES

The examples that follow are intended in no way to limit the scope of this invention but instead are provided to illustrate representative embodiments of the present invention. Many other embodiments of this invention will be apparent to one skilled in the art.

Hypothalamic Tissue from Rat Brain.

Extraction procedures of hypothalamic tissue have been previously described in detail (for example, see Imbery et al., (2008) Brain Res., 1193, p. 93-101). Briefly, brain tissue sections containing the DMH and POA were prepared from male Sprague-Dawley rats (Harlan; 100-150 g) that were housed under standard conditions and provided food and water ad libitum. Before each session, a rat was anesthetized using isoflurane and promptly decapitated. After dissection of the brain, a tissue block containing the hypothalamus was mounted on a vibratome and bathed in artificial cerebral spinal fluid (aCSF). Sagittal plane, 400-μm-thick tissue sections were produced and then placed in a submersion recording chamber.

Tissue Perfusion and Probe Injection.

Tissue sections were continually perfused with normal aCSF, which consisted of (in mM): 124 NaCl, 26 NaHCO3, 10 glucose, 5 KCl, 2.4 CaCl2, 1.3 MgSO4, and 1.24 KH2PO4. After gentle aeration (95% O2, 5% CO2), the aCSF (300 mOsM; pH 7.5) was allowed to gravity flow at 1-2 ml/min into the recording chamber (volume=2 ml). Approximately 1-5 μl of probe solution (AuNPs in bovine serum albumin suspension) were backfilled into glass microelectrodes pulled to a tip diameter of ˜2-5 μm. The solution was pressure-injected into the tissue area of interest, using a nitrogen puffer system as is known in the art. Successful injection sites aimed at the DMH, regardless of probe type, were designated as ‘target’ tissue (n=57), while non-DMH were ‘control’ (n=7). Tissue classified as non-DMH included injection sites in the AHA or the ventromedial hypothalamus (VMH). Probes were allowed to diffuse through the tissue for an average of 5.9±0.1 h at a mean temperature of 35.5±0.18° C. A thermocouple was placed adjacent to the tissue slices to constantly monitor the temperature.

Histology.

Upon removal from the recording chamber, the tissue sections were allowed to soak in 30% paraformaldehyde/sucrose solution at 12° C. for a minimum of 3 h. Tissue was frozen and sliced into 50-μm-thick sections using a microtome (Leica SM-2000R). After suspension in phosphate-buffered saline, the tissue sections were mounted on slides and were given 12 h to dry at room temperature (RT) before beginning the staining process. Slides were first placed in a 50:50 chloroform-alcohol solution for 3 h at RT. Afterwards, they were carried through a series of rehydration steps, for 5 minutes each in 300 ml of, successively, 95.5% reagent alcohol, 70% reagent alcohol, and distilled water, followed by a 5-min submersion in phosphate buffer monobasic solution and 4 min in Giemsa stain. Excess stain was then removed via 2 minutes of agitation in 95.5% reagent alcohol before the slides were placed in 300 ml of xylene (Sigma; >98.5%) for 12 h. Slides were cover-slipped and allowed 12 h to set before microscopy.

Probe Visualization and Quantification.

The location and confirmation of each injection site were noted on a section diagram adapted from a rat brain atlas (see Pellegrino et al., 1979, A Stereotaxic Atlas of the Rat Brain, Plenum Press, New York). Quantification of the probes involved tallies of labeled neurons and their location. Labeled neurons were readily identified by the presence of AuNPs in their Giemsa-stained somas, which can be visualized under normal bright field illumination, and then confirmed through their reflective properties under dark field illumination. To reduce bias, counters were blinded to the tissue sample's probe treatment. Microscopy images were obtained using an Olympus CCD camera (DP11) at 4×, 10×, and 40× magnification. Full-image brightness and contrast were non-destructively adjusted using Adobe Photoshop.

Statistical Analysis.

Mann-Whitney U-tests were conducted when comparing two treatment groups, whereas a Kruskal-Wallis test was utilized for comparing three or more treatment groups. To determine which factors influence the quantity of labeled neurons within the POA, a principal components analysis (PCA) was used to develop a multiple regression model. Robust regression confidence intervals and standard errors were generated using bootstrap methods. These estimates were bias-corrected and validated using a separate jackknife procedure (See Efron, 1977, Bootstrap Methods: Another Look at the Jackknife, Vol. 7, Institute of Mathematical Studies). P values of less than 0.05 were stated as significant.

Experimental Results.

Upon tissue placement into the chamber, a small concentration of gold nanoprobes (“AuNPs”) was pressure-injected into the DMH. These AuNPs are spherical, colloidal nanoparticles composed of a dielectric silica core and an ultrathin metallic (gold) coating, providing them with a strong reflectivity (peak frequency=524 nm) and an extremely low biotoxicity. The AuNPs were conjugated with (i) a polyclonal antibody for the rat vesicular glutamate transporter type-2 (VGLUT-2; Millipore) and (ii) PEI. This fully conjugated nanoprobe complex, consisting of both V-GLUT-2 and PEI antibodies attached onto a gold nanoshell (diameter=5 nm), was designated as probe A.

After injection into the tissue slice, specific antibody binding of probe A to nearby VGLUT-2s (which are exposed to the terminal surface during vesicle formation) facilitates AuNP uptake exclusively into synaptic vesicles at axon terminals (Jung et al., 2006). Once inside a newly formed vesicle, “endosomal escape” occurs when the attached, cationic PEI conjugates attract water molecules into the vesicular lumen, initiating a progressive “proton sponge effect”. Upon hydrosaturation, the vesicles lyse and a significant number of AuNPs diffuse into the cytosol. Over the next several hours, while the tissue slice equilibrates to the chamber environment, some AuNPs will retrogradely diffuse from the synaptic terminal region to the neuronal soma (see, for example, Bergen et al., 2008, “Nonviral approaches for neuronal delivery of nucleic acids”, Pharm. Res. 25, p. 983-998; and Suk et al., 2007, “Quantifying the intracellular transport of viral and nonviral gene vectors in primary neurons”, Exp. Biol. Med. (Maywood) 231, p. 461-469).

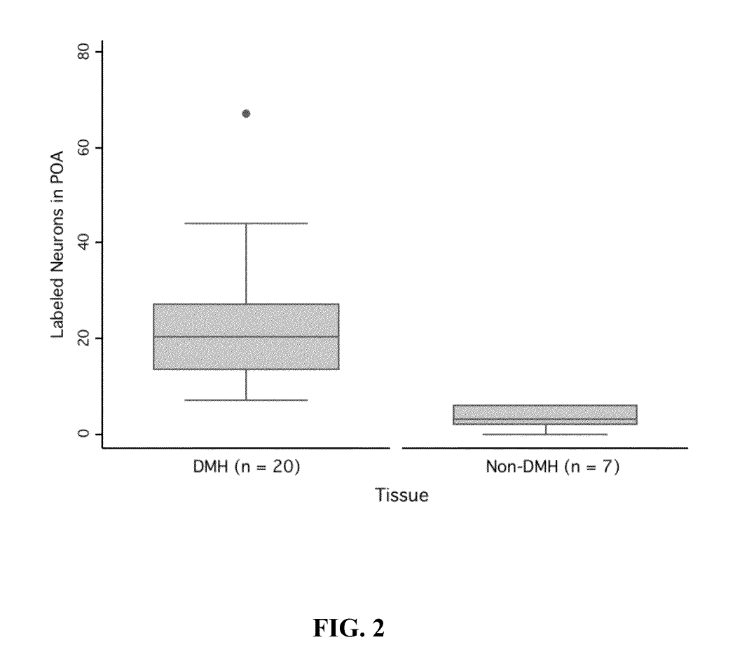

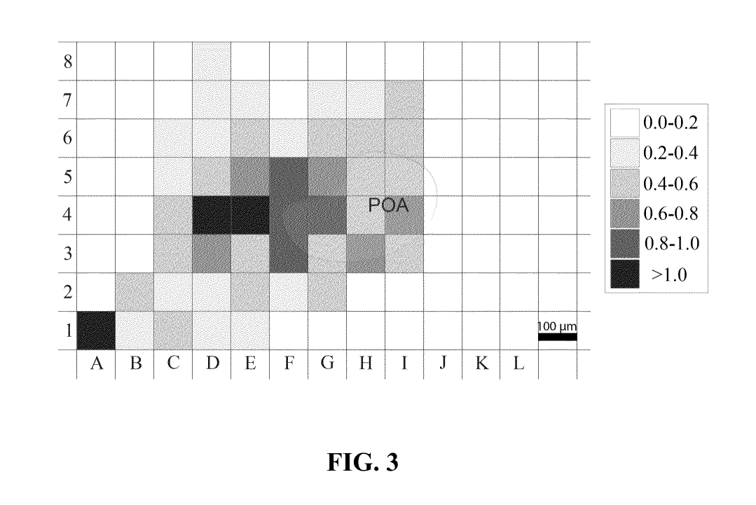

Trials assessing the efficacy of probe A yielded results dependent on initial probe injection location. Significant differences in somatic labeling were observed between tissues injected in the DMH as compared to non-DMH tissues, as shown in FIG. 2, wherein probe injection into the DMH translated into significantly higher POA somatic labeling. DMH-injected tissues displayed a relatively higher variance in POA labeling compared to non-DMH injected tissues, with at least one sample exhibiting over 60 labeled soma. Discrete reflecting units within clearly marked somas were observed. Observed reflectances do not represent individual nanoprobe complexes since, individually, these particles have insufficient surface area to reflect the necessary amount of perceivable light. Instead, it is likely that we perceived collections of several probes that can be characterized as nanoaggregates. An adjusted density map was constructed to characterize probe A's spatial distribution in DMH-injected tissue (n=20). In tissue slices with confirmed injection sites in the DMH, the majority of labeled cells were located in the POA. Not surprisingly, the highest densities (excluding areas immediately surrounding the injection site) were observed in the rostral sections of the POA—those areas closest to the DMH, as shown in FIG. 3. In contrast, control tissue with injection sites to the mammillary peduncle or anterior hypothalamic area (AHA) showed significantly lower POA somatic labeling. Outside the POA, probes were relatively scattered. Soma closer to the injection site reporting higher probe accumulations may possibly reflect shorter traveling times.

To determine the differential contribution of the complex's components, isolation of individual component effects was required. To accomplish this task, progressively simpler, control probes were designed by removing individual modifications (all probes were synthesized by Nanopartz, Inc. of Loveland, Colo., www.nanopartz.com). After testing the performance of the complete probe (probe A; AuNP+VGLUT-2 antibody+PEI), we studied the properties of probe B (AuNP+VGLUT-2 antibody only), probe C (AuNP+PEI only), and probe D (AuNP shell only, diameter=5 nm). A linear multiple regression model was developed to determine the marginal effect of probe type, location of injection site, diffusion time, and perfusion temperature, respectively, on the number of POA neurons labeled. All explanatory variables were assessed for co-linearity, and all showed sufficient independence. The regression model significantly explained approximately 50% of the POA labeling variation. Bootstrapped linear regression models (n=45, iterations=10,000) indicate that overall POA labeling was significantly affected by probe type and injection site (see Table 1 below). Injection in the DMH yielded, on average, significantly more POA labeling after controlling for probe type and other factors. Although diffusion time did not show significance at the 0.05 level (most tissue slices were perfused for approximately 6 hours with minimal variation), future investigation and manipulation of this variable may be informative.

TABLE 1
Labeling Efficacy by Probe Configuration

| | | | | 95% | 95% |
|---|---|---|---|---|---|
| | | Bootstrap | | Conf. | Conf. |
| | | Standard | | Interval | Interval |
| | Beta | Error | Significance | (lower) | (upper) |
| Neither | 0 | 5.4 | 1.00 | −10.5 | 10.5 |
| PEI only | −6 | 4.9 | 0.21 | −15.6 | 3.5 |
| VGLUT-2 only | 2.3 | 5.1 | 0.65 | −7.6 | 12.3 |
| Both | 11.6 | 5.4 | 0.03 | 1.0 | 22.3 |
| Tissue Type | 11.7 | 3.4 | 0.001 | 5.0 | 18.5 |
| Time (min) | −0.2 | 0.1 | 0.07 | −0.3 | 0.0 |
| Temperature | 0.7 | 1.4 | 0.65 | −2.1 | 3.4 |
| (° C.) | | | | | |

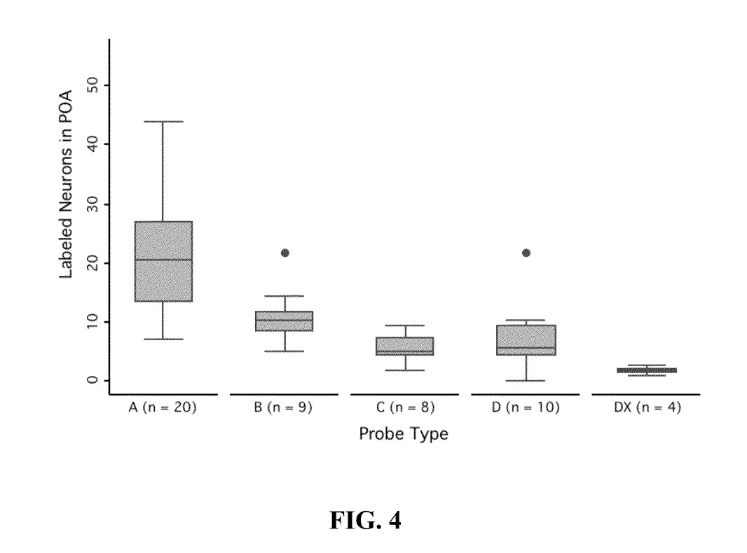

The regression model described above also estimated the marginal effect of individual conjugates on POA labeling efficacy, where progressive deconjugation decreased overall labeling efficiency, as observed in FIG. 4. When compared to baseline performance of the “naked” 5-nm probe, the average increase/decrease in POA neurons labeled was calculated by conjugate type, statistically controlling for other factors. While adding either VGLUT-2 or PEI individually (but not together) had some impact on the efficacy of the probe, only the attachment and interaction of both conjugates significantly improved POA labeling over baseline. It was also noted that in tissues treated with control probes (especially unconjugated ones), somatic labeling was generally more sporadic and less specific to any particular region of the tissue.

Because unconjugated gold nanoprobes (e.g., probe D) are, by design, biocompatible and inert, they are unlikely to initiate the cell's immunological response which would subject them to degradation. This property, when coupled with their miniscule size, makes “naked” nanoprobes more likely to pass through the synaptic machinery than their larger, conjugated counterparts (see Verma et al., 2008, “Surface-structure-regulated cell-membrane penetration by monolayer-protected nanoparticles”, Nat. Mater., 7, p. 588-595). To determine the effect of nanoprobe size, we enlarged the “naked” shells tenfold and compared the performance of these extra-large probes (designated as probe DX, diameter ˜47 nm; peak reflectance=530 nm; obtained from Nanopartz, Inc., www.nanopartz.com) with their 5-nm counterparts. Fifty nanometers was chosen because it is at the uppermost limits of synaptic vesicle diameters and most synaptic vesicles do not grow to be nearly this size. X² Comparisons between probes D and DX yielded a significant difference, with probe DX showing decreased POA labeling efficacy (see Table 2 below).

TABLE 2
Chi-square results by probe configuration or injection site.

| | Probe A | | | | |
|---|---|---|---|---|---|
| | (non-DMH; | | | | |
| | AuNP + | Probe B | Probe C | Probe D | Probe DX |
| Probe | VGLUT-2 + | (AuNP + | (AuNP + | (AuNP, | (AuNP, |
| Type | PEI) | VGLUT-2) | PEI) | 5 nm) | 50 nm) |
| A | 15.03 | 5.68 | 14.57 | 11.8 | |
| (DMH) | | | | | |
| B | | | 6.58 | 3.71 | |
| C | | | | 0.29 | |
| D | | | | | 5.14 |

Probe DX performed even less efficiently than all its 5-nm deconjugated/unconjugated counterparts, registering a similar POA labeling efficacy to non-DMH probe A samples (see FIG. 4). Accordingly, in addition to initial injection site and conjugate type, size may be another major factor effecting probe uptake. Differences between probes D and DX's spatial distribution were not apparent on recorded anatomical data.

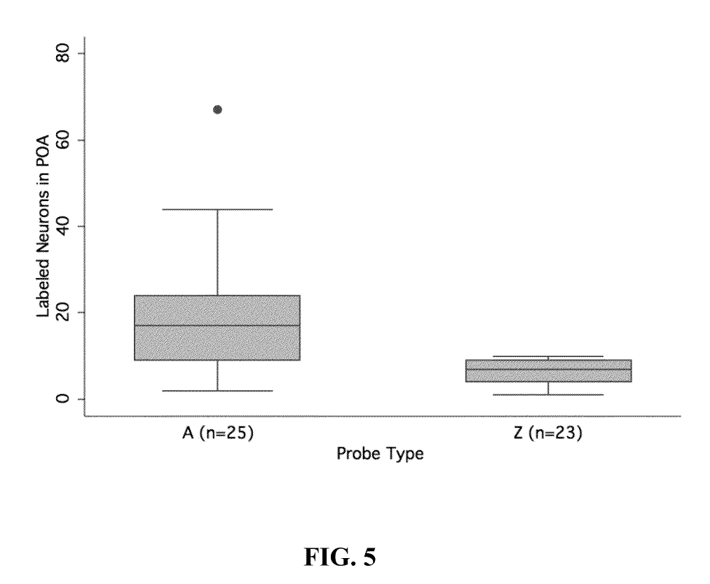

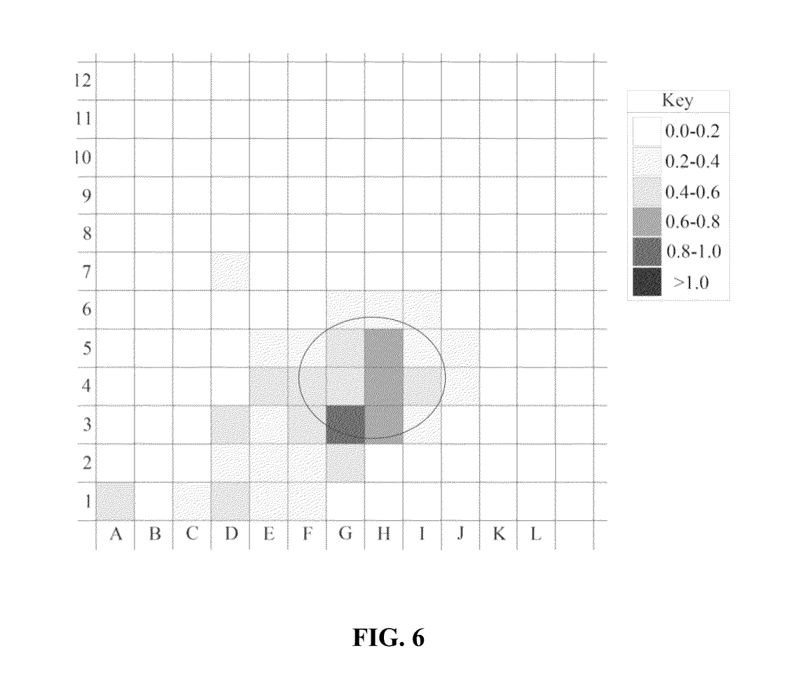

Subsequent trials assessed the efficacy of the GABAergic probes (Probe Z; n=23), which yielded significantly lower POA labeling when compared to fully-conjugated glutamatergic probes (p<0.001), despite statistically similar perfusion temperature, diffusion time, and initial injection volume (see FIG. 5). Spatial distribution patterns using Probe Z (AuNP+VGAT antibody+PEI) were similar to those using Probe A (AuNP+VGLUT-2 antibody+PEI), where the densest labeling occurs in the anterior POA. The trials using Probe Z differed, however, in having lower overall labeling, which is reflected by lighter density shades in FIG. 6.

Discussion of Experimental Results.

The mechanisms by which the nervous system maintains or adjusts body temperature in response to thermal stressors remain a fundamental pursuit in physiology. Recent studies suggest that responses, such as hyperthermia, can be elicited by activation of neurons in the DMH, which receive direct input from thermally responsive neurons in the POA. Therefore, to produce distinct changes in thermoregulatory control, these POA neurons may have direct axonal connections to the DMH. Phenotypical and connectivity assessments of neuronal populations within these areas could provide insight to their functional physiology. Since VGLUT expression acts as a specific biomarker of a neuron's glutamatergic phenotype, it is a sufficient indicator of the glutamatergic machinery's presence within the terminal. Of the three known neuronal VGLUT isoforms, hypothalamic synapses predominantly express VGLUT-2. Therefore, it is reasonable to conclude that probes with the VGLUT-2 antibody are preferentially binding to and being absorbed in glutamatergic axon terminals. By observing and quantifying neurons labeled with probe A, we were able to simultaneously ascertain a likely glutamatergic phenotype, while confirming connections between the DMH and the POA.

To understand the marginal contribution of the probe's constituent parts, it is instructive to elaborate on the mechanisms by which they affect efficacy rates. Terminal uptake and endosomal escape can be classified in two ways: facilitated and passive. During vesicular uptake, probes with the VGLUT-2 antibodies (probes A and B) were more likely to enter and remain with the vesicles because they are more likely to bind to exposed VGLUT-2s and be co-transported into the lumen upon vesicular formation. Although still possible through passive diffusion, non-VGLUT-2 probes (C and D) are less likely to be actively taken into vesicles.

Once inside the vesicular lumen, the probe's fate is codetermined with that of the vesicle. Excluding degradation, three proposed, post-uptake vesicular mechanisms may affect probe efficacy. Vesicles may exist in a transient kiss-and-run cycle, a short-term recycling pool, or a long-term reserve pool.

Although the molecular processes underlying the kiss-and-run recycling mechanism are still unclear, this pathway is too transient for any appreciable probe vesicular entry; it may even return probes into the synaptic cleft. It is therefore unlikely to be a starting point for probes that reach the soma. Vesicles that follow the short-term recycling pool or long-term reserve pool pathway exist long enough to be reasonable probe entry points. Probes that display PEI conjugates (probes A and C) take advantage of the “proton sponge effect” which facilitates their escape into the cytosol. Non-PEI probes (probes B and D) are less likely to exit intact vesicles because the vesicular plasma membrane presents a significant barrier. Because of this physical obstruction, non-PEI probes can presumably exit into the cytosol in appreciable amounts only when the vesicle itself and integral membrane proteins are lysed and degraded by the cellular machinery. Based on the relative absence of lysosomes within the presynaptic terminal, integral vesicular membrane proteins are retrogradely transported to the soma, where they are most likely destroyed. In this sense, and without wishing to be bound by theory, the similarities in the efficacy rates of probes B and C (see FIG. 4) can be explained in the following way: while probes with the VGLUT-2 antibody are more likely to remain within the vesicles and therefore be present in larger numbers after formation, only a small percentage escape passively into the cytosol and reach the soma, presumably through vesicle degradation. For PEI-conjugated probes, the reverse is true: while relatively fewer of them passively remain in the vesicle after formation, those that do remain in the vesicle after formation are more likely to actively escape into the cytosol and diffuse to the soma through vesicle osmolysis. Furthermore, the absence of the VGLUT-2 antibody enables these particular probes to nonspecifically enter neurons. While it is clear that possessing either conjugate may be sufficient to reach the soma, efficacy rates are significantly higher and more specific when both are present.

Although conjugate type plays a significant role, overall size may also influence probe efficacy. While adding the appropriate conjugates facilitates probe specificity, it also adds to the probe complex's size. The larger the size, the more likely steric effects (such as blockage or entanglement) may influence efficacy. For example, despite probe D's lack of any facilitative conjugates, tissue injected with this “naked” probe shows a comparable efficacy rate to its deconjugated counterpart (see Table 2), which may be attributable to this steric effect.

Concurrently, we also observe that merely increasing the probe shell size, as in the case of probe DX, is sufficient to mitigate efficacy rates. When coupled with its biocompatibility, the unconjugated 5-nm probes take advantage of various mechanisms to enter neurons, such as occasionally passing interstitially through the plasma membrane, which generally cannot be achieved by their conjugated counterparts (see, for example, Verma et al., 2008, Nat. Mater., 7, p. 588-595). Accordingly, we observe a trade-off between specificity and mobility.

Although a median of approximately 20 labeled neurons, from a population of thousands within the POA, may seem small, this number only represents labeling from intact neurons within a single 400-μm section, during a time course of only a few hours. Furthermore, when considering the number of oblique, axonal fibers severed during the slicing process, labeling of 60 or more neurons (like the outlier in FIG. 2) is still possible. This technology has demonstrated its versatility by allowing conjugation of glutamatergic antibodies, targeting one of the two most abundant neurotransmitter systems in the central nervous system. Countless conjugate permutations could allow for specific visualization, customized characterization, and electrophysiological recording of hypothalamic cells and other neuronal populations.

Nanoprobes conjugated to both (1) PEI and (2) antibodies to vesicular transporter proteins were effective in labeling POA neurons, although there were differences depending on the antibodies that were used. Probe Z (conjugated to VGAT antibody) labeled POA neurons at a significantly lower efficacy rate than was obtained with Probe A (conjugated to VGLUT antibody).

Several factors explain this difference, which could be viewed as surprising given that the GABAergic phenotype comprises the majority of hypothalamic neurons (−60%) and thus one might otherwise expect greater labeling from Probe Z relative to Probe A. From a chemical standpoint, VGAT's binding potential to its endogenous ligand (GABA) is different from VGLUT-2's interaction with its endogenous ligand (glutamate). If the principle of a “molecular velcro” does apply to the facilitated vesicular entry of these probes, then it is reasonable to assume that the differential binding kinetics between these two transporters may affect the binding interactions with their respective probes. This, in turn, alters the likelihood of vesicular uptake and therefore, somatic labeling. Furthermore, at physiological temperatures (−36° C.), glutamate is endocytosed at faster rates and in larger vesicular compartments. These differences may be related to glutamate's general metabolic role in neuronal cells, as opposed to GABA's more specialized, neurotransmitter capacity.

### INCORPORATION BY REFERENCE

All publications, patents, and patent applications cited herein are hereby expressly incorporated by reference in their entirety and for all purposes to the same extent as if each was so individually denoted.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

The articles “a” and “an” are used herein to refer to one or to more than one (i.e. to at least one) of the grammatical object of the article. By way of example, “a nanoprobe” means one nanoprobe or more than one nanoprobe.

Any ranges cited herein are inclusive.

1. A neural nanoprobe comprising metallic nanoparticles, wherein said metallic nanoparticles are conjugated to both a cationic polymer and an antibody to a vesicular transport protein.

2. The neural nanoprobe of claim 1, wherein said metallic nanoparticles comprise a dielectric silica core with a gold coating.

3. The neural nanoprobe of claim 1, wherein said antibody is a polyclonal antibody.

4. The neural nanoprobe of claim 1, wherein said vesicular transport protein is a transporter of a neurotransmitter selected from the group consisting of glutamate and GABA.

5. The neural nanoprobe of claim 4, wherein said vesicular transport protein is a vesicular glutamate transporter protein.

6. The neural nanoprobe of claim 5, wherein said antibody to said vesicular glutamate transporter protein is the rat vesicular glutamate transporter type-2 antibody.

7. The neural nanoprobe of claim 4, wherein said vesicular transporter protein comprises a vesicular GABA transporter protein.

8. The neural nanoprobe of claim 1, wherein the diameter of said metallic nanoparticles is less than 7 nm.

9. The neural nanoprobe of claim 1, wherein said cationic polymer is polyethylenimine.

10. A method for retrograde labeling of neurons comprising:
injecting neural nanoprobes into neural tissue;
allowing uptake of said neural nanoprobes into synaptic vesicles at axonal terminals;
allowing endosomal escape of said neural nanoprobes into the cytosol; and
allowing diffusion of said neural nanoprobes from the synaptic terminal region to the neuronal soma;
wherein said neural nanoprobes comprise metallic nanoparticles conjugated to both a cationic polymer and an antibody to a vesicular transport protein.

11. The method of claim 10, wherein said neural tissue comprises an in vitro slice of neural tissue.

12. The method of claim 10, wherein said antibody to a vesicular transport protein is the rat vesicular glutamate transporter type-2 antibody.

13. The method of claim 10, wherein said cationic polymer is polyethylenimine.

14. The method of claim 10, wherein said vesicular transport protein is a transporter of a neurotransmitter selected from the group consisting of glutamate and GABA.

15. The method of claim 14, wherein said vesicular transport protein is a vesicular glutamate transporter protein.

16. A kit for retrograde labeling of neurons comprising:
metallic nanoparticles conjugated to both a cationic polymer and an antibody to a vesicular transport protein; and
instructions for delivering said conjugated metallic nanoparticles to neural tissue such that somatic labeling can occur.

17. The kit of claim 16, wherein said metallic nanoparticles comprise a dielectric silica core with an ultrathin gold coating.

18. The kit of claim 16, wherein said cationic polymer is polyethylenimine, and wherein greater than one polyethylenimine chain is conjugated per metallic nanoparticle.

19. The kit of claim 16, wherein said vesicular transport protein is a transporter of a neurotransmitter selected from the group consisting of glutamate and GABA.

20. The kit of claim 19, wherein said vesicular transport protein is a vesicular glutamate transporter protein.
